(19) United States
(12) Patent Application Publication
(10) Pub. No.: US 2004/0146468 A1
(43) Pub. Date: Jul. 29, 2004

(54) Oral compositon comprising an extract from the bark of albizzia myrioplylla

(76) Inventors: Jean Daniel, Vic-le-Comte (FR); Leon Cariel, Paris (FR)

Correspondence Address: BIRCH STEWART KOLASCH & BIRCH, PO BOX 747, FALLS CHURCH, VA, 22040-0747, US

(21) Appl. No.: 10/479,116

(22) PCT Filed: May 30, 2002
(86) PCT No.: PCT/FR02/01825

(30) Foreign Application Priority Data
May 30, 2001 (FR) 0107063

(51) Int. Cl. A61K 7/26; A61K 35/78

(52) U.S. Cl. 424/58; 424/775

(57) ABSTRACT

The present invention provides an oral composition comprising an effective quantity of an Albizzia myriophylla bark extract to suppress or reduce the gustatory function, in particular as regards a bitter and/or sweet and/or acid taste, advantageously a bitter and/or sweet taste, and a suitable excipient. It also provides the use of an Albizzia myriophylla bark extract or of an oral composition in accordance with the invention, to suppress or reduce the gustatory function. Finally, it provides an Albizzia myriophylla bark extract or a composition in accordance with the invention as a drug, in particular intended for the treatment of bulimia, a compulsive eating disorder, alcohol and/or nicotine dependence obesity and for the prevention of obesity.

The present invention relates to substances that suppress or reduce the gustatory function, in particular as regards a bitter and/or sweet taste. In particular, it relates to the use of an Albizzia myriophylla bark extract to suppress or reduce the gustatory function, and to compositions including said extract.

Products that suppress the gustatory function are highly desirable for the following reasons:

A wide variety of bad-tasting foodstuffs, drinks, or pharmaceutical compositions for oral administration are known. In particular, many pharmaceutical compositions taste bad and their oral administration may be accompanied by certain difficulties or problems. A large number of pharmaceutically active agents leave a bitter taste in the mouth, either during or immediately after their oral administration. They include the following active agents: acetaminophen, ampicillin, azithromycin, chlorpheniramine, cimetidine, dextromethorphan, diphenhydramine, erythromycin, ibuprofen, phenylbutazone, psuedoephedrine, ranitidine, spironolactone, and theophylline. The bitter taste of those active agents in a liquid suspension is inevitably detected during its ingestion or immediately after ingestion. Further, the bitter taste in a tablet, a capsule, a suspension, or other oral dosage forms can also be detected during administration if the bitter agent is brought into contact with the taste buds in the mouth, for example because the dose has been in the mouth too long, because of unintentional chewing, or because of other reasons for releasing the bitter pharmaceutical agent into the mouth.

The oral administration route is generally the preferred route for many pharmaceutical agents such as those described above, as it allows administration that is easy and cheap. However, the co-operation of the patient is sometimes an important factor since the patient must swallow a tablet, capsule, or suspension. Patients give many reasons for refusing or being incapable of accepting oral administration of a drug, such as unattractive presentation, too large a size, bad taste, or simply fear that a particular dosage form might become stuck in the throat. Some patients, in particular children and old people, refuse solid oral forms for administering pharmaceutical preparations because of difficulties with ingestion. For that reason, active principles are frequently administered in the form of a syrup or a tablet that can be chewed. However, the pharmaceutical agent often has a bitter taste which is so strong that it cannot be used in a syrup nor can it be chewed, and that taste prevents patients from self-administering that form of dosage orally. As a result, it is important to mask the bitter taste of pharmaceutical agents so that it is reduced or suppressed in any oral dosage form.

Conventionally, sweetening or flavoring agents have been used to mask the taste of such compositions. Such agents are intended to cover the bad taste of the composition with another taste which, it is hoped, is sufficiently intense to hide any bitter taste. That technique works well in the case of pharmaceutical products having a slightly bitter taste, but is not sufficient to mask strong bitter tastes.

Other alternative approaches that have been used to mask the bitter taste of certain pharmaceutical products include microencapsulation of the active agent having the bad taste or coating it, for example with ethylcellulose or mixtures of ethylcellulose and hydroxymethylcellulose or other cellulose derivatives. However, such prior art products suffer from a disadvantage due to the fact that coating polymers release the active agent inconsistently and cannot release it immediately or at the right time. Further, the use of such cellulose derivatives is often insufficient to mask the taste of a particularly bitter active agent.

It is also known that the active pharmaceutical compound can be chemically modified to cause it to lose its bitter taste. However, such a method can only be used for very specific compounds.

Adding a lipid is also well known (Japanese patents JP-A-55-8956 and JP-A-62-265234). Those methods are all insufficient as regards producing the desired suppression of the bitter taste.

Finally, it is also known that gymnemic acid extracted from leaves of Gymnema Sylvestre can be used to mask the bitterness of such compositions.

That acid is also known for suppressing a sweet taste (Hellekant et al, Physiology & Behavior, vol 60, n° 2, 469-479, 1996) and it is used in alcohol and tobacco withdrawal, in the prevention of obesity, in particular in diabetics who have to limit their calorie intake and thus go on diets, and for the treatment of people with problems with bulimia and compulsive eating disorders (U.S. Pat. No. 5,605,698).

Reducing the sweet taste of food decreases the level of satisfaction or the desire to eat. Thus, reducing the sensitivity to sugar spontaneously tempers the tendency to eat between meals and effortlessly reduces the desire to consume alcohol or to smoke, which is not the case with traditional methods. The normal solution proposed for reducing calorie intake is to reduce the sugar and lipids intake helped, for example, by taking low calorie food substitutes. However, strong willpower is required as preventing obesity and limiting calorie intake necessitates making an effort at every mealtime, every day. Such self-control frequently causes fresh frustrations.

However, the disadvantages of gymnemic acid remain its procurement, the process for its preparation (separation and purification), its chemical stability and its price.

As a result, there is still a need to find novel products that can reduce or suppress the gustatory function, in particular the bad taste of certain chemical products and the sweet taste of foodstuffs.

Surprisingly, the present inventors have discovered that an Albizzia myriophylla bark extract can completely suppress or reduce the gustatory function for a period of three or four hours following ingestion. This tree, native to provinces in South Vietnam and named “Cay Song Ran”, has been used as a substitute for licorice because of the sweet taste of its bark and the ross from its rhizome. Research has been carried out to isolate the molecule responsible for that sweet taste and glucosides and lignanes as well as an alkaloid contained in bark extracts from that tree have been discovered (Ito et al Phytochemistry, vol 37, N° 5, 1455-1458 and Ito et al., Chem. Pharm. Bull., 42 (9), 1966-1967, 1994).

However, those prior art documents neither mention nor suggest effects that plant has on the suppression or reduction of the gustatory function.

Thus, the present invention provides an oral composition comprising an effective quantity of an Albizzia myriophylla bark extract to suppress or reduce the gustatory function, in particular as regards a bitter and/or sweet and/or acid taste, advantageously as regards a bitter and/or sweet taste, more advantageously as regards a sweet and/or acid taste, and a suitable excipient.

Advantageously, the quantity of Albizzia myriophylla bark extract present in said composition can suppress or reduce the gustatory function for 2 hours (h) to 4 h after ingestion thereof.

Advantageously, the Albizzia myriophylla bark extract is an extract from branch bark, in particular a dry extract, advantageously purified.

Advantageously, it is a protein extract. It appears to be a protein which is the active product of such an extract. Advantageously, said protein has a molecular weight of about 50,000 D.

More advantageously, the bark extract is obtained using a method that comprises the following steps:

a) pulverizing Albizzia myriophylla branch bark;

b) extracting one or more times, advantageously three times, the pulverized bark obtained in step a) in a solvent that is miscible with water, advantageously selected from alcohol and water and mixtures thereof in any proportions; more advantageously, the alcohol is selected from methanol or ethanol;

c) filtering the solution obtained in step b) and eliminating the solvent, advantageously by evaporation.

More advantageously, the composition also comprises a flavoring agent; in particular, the flavor is selected from the group constituted by cherry, strawberry, orange, chocolate, cola, vanilla, grape, mint, and lemon.

Advantageously, the composition is in the form of a syrup, a suspension, chewing gum, a water-dispersible powder, an effervescent tablet, or granules, advantageously chewing gum.

When a solid composition is prepared in the form of tablets, the Albizzia myriophylla bark extract is mixed with a pharmaceutical vehicle such as gelatin, starch, lactose, magnesium stearate, talc, gum Arabic, or the like. The tablets can be coated with saccharose or other suitable materials or they can be treated so that they have a long-term or slow-release activity and so that they continuously release a predetermined quantity of active principle.

A capsule preparation is obtained by mixing the Albizzia myriophylla bark extract with a diluent and pouring the mixture obtained into soft or hard capsules or gelules.

A preparation in the form of a syrup or elixir can contain the Albizzia myriophylla bark extract together with a sweetener, an antiseptic, and an agent producing flavor and a suitable colorant.

The water-dispersible powders or granules can contain the Albizzia myriophylla bark extract with dispersion agents or wetting agents, or suspension agents, as well as taste correctors or sweeteners.

The quantity of Albizzia myriophylla bark extract present in the oral dosage forms depends on the desired effect, reduction or suppression of the gustatory function, and the time for which that effect is to last (several minutes to several hours).

In a particular embodiment, said composition also comprises a bad tasting substance, and the quantity of Albizzia myriophylla bark extract present in said composition can reduce or suppress said bad taste, which is due in particular to being too bitter and/or too acid.

Advantageously, said composition is a pharmaceutical composition intended for oral administration, a foodstuff, a drink, a toothpaste, or a solution for use in the mouth, and more advantageously a pharmaceutical composition intended for oral administration.

Advantageously, the substance with the bad taste is an active agent selected from the group formed by acetaminophen, ampicillin, azithromycin, chlorpheniramine, cimetidine, dextromethorphan, diphenhydramine, erythromycin, ibuprofen, phenylbutazone, psuedoephedrine, ranitidine, spironolactone, and theophylline

The present invention also provides the use of an Albizzia myriophylla bark extract or a composition in accordance with the present invention to suppress or reduce the gustatory function, in particular as regards bitterness and/or a sweet taste and/or acidity, advantageously as regards bitterness or a sweet taste, and more advantageously regards a sweet taste and/or acidity.

Advantageously, it concerns its use as a hunger suppressant, as a slimming aid or to avoid weight gain and as an alcohol and tobacco withdrawal aid.

The present invention also provides a method of reducing or suppressing the taste of a composition, characterized in that it comprises adding an effective quantity of an Albizzia myriophylla bark extract to said composition.

It also provides a method of reducing or suppressing the gustatory function, in particular as regards a bitter and/or sweet and/or acid taste, advantageously as regards a bitter and/or sweet taste, and more advantageously as regards a sweet and/or acid taste, characterized in that it comprises oral administration of an effective quantity of an Albizzia myriophylla extract or of a composition in accordance with the present invention.

In a particular implementation of said method, a composition the taste of which is to be suppressed or reduced is administered at the same time or after the Albizzia myriophylla bark extract.

The present invention also provides an Albizzia myriophylla bark extract and an oral composition in accordance with the invention as a drug, advantageously for the treatment of obesity, bulimia, a compulsive eating disorder, alcohol and/or nicotine dependence, and to prevent obesity.

Non-limiting examples for the preparation of the extract of the invention will now be given.

### EXAMPLE 1

1 kilogram (kg) of Albizzia myriophylla branch bark was pulverized coarsely. It was extracted by decoction under reflux in 10 liters of 50% ethanol for one hour. It was filtered to recover the extractive solution. The operation was repeated twice more using the plant material recovered after filtering. The extractive solutions were combined and evaporated to dryness under reduced pressure. 142 grams (9) of dry extract was obtained.

### EXAMPLE 2

1 kg of Albizzia myriophylla branch bark was pulverized coarsely. It was extracted by decoction under reflux in 7 liters of pure methanol for one hour. It was filtered to recover the extractive solution. The operation was repeated twice more using the plant material recovered after filtering. The extractive solutions were combined and evaporated to dryness under reduced pressure. 107 g of dry extract was obtained.

### EXAMPLE 3

1 kg of Albizzia myriophylla branch bark was pulverized coarsely. It was extracted by decoction under reflux in 10 liters of water for one hour. It was filtered to recover the extractive solution. The operation was repeated twice more using the plant material recovered after filtering. The extractive solutions were combined and concentrated to 1 liter under reduced pressure. The concentrated solution was then freeze dried. 172 g of freeze dried extract was obtained.

### EXAMPLE 4

A protein with a strong sweetening and taste inhibiting power with a residual effect was obtained. 1 kg of Albizzia myriophylla twig bark was coarsely ground and allowed to extract by maceration in water at 45° C. for 12 hours.

The operation was repeated three times more. The extractive solutions were combined and concentrated under reduced pressure at 40° C.

The concentrated solution was dialyzed against distilled water through a semi-permeable membrane with a cutoff threshold of 25,000 D.

The solution retained on the membrane was then filtered through paper to eliminate the insoluble matter which had formed during dialysis.

This concentrated solution was then deposited on a Cephadex G 50 gel column.

It was eluted with distilled water and the eluted fraction with a volume in the range 4.5 to 4.7 times the dead volume of the column was recovered.

This fraction was freeze dried.

Colorimetric analysis using Coomassie Blue revealed a total protein content of close to 100%.

This fraction underwent electrophoresis separation to reveal a major band corresponding to a molecular weight of 50,000 D.

This fraction had a sweetening power 77 times that of saccharose, measured by successive dilution and gustatory tests.

Further, after being deposited on the tongues of volunteer human subjects, as before, the fraction obtained caused, complete suppression of the taste for sweet and acid flavors which persisted for more than two hours.

1. An oral composition comprising an effective quantity of an Albizzia myriophylla bark extract to suppress or reduce the gustatory function, in particular as regards a bitter and/or sweet and/or acid taste, and a suitable excipient.

2. An oral composition according to claim 1, characterized in that the quantity of Albizzia myriophylla bark extract present in said composition can suppress or reduce the gustatory function of the tongue for 2 to 4 hours after ingestion thereof.

3. A composition according to claim 1 or claim 2, characterized in that it further comprises a substance with a bad taste and in that the effective quantity of the Albizzia myriophylla bark extract present in the composition can reduce or suppress said bad taste.

4. A composition according to claim 3, characterized in that the bad taste is due to too much bitterness and/or too much acidity.

5. A composition according to any one of claims 1 to 4, characterized in that it is a pharmaceutical composition intended for oral administration, or a foodstuff, or a drink, or a toothpaste or a solution for use in the mouth.

6. A composition according to claim 5, characterized in that it is a pharmaceutical composition intended for oral administration.

7. A composition according to claim 6, characterized in that the substance with the bad taste is an active agent selected from the group constituted by acetaminophen, ampicillin, azithromycin, chlorpheniramine, cimetidine, dextromethorphan, diphenhydramine, erythromycin, ibuprofen, phenylbutazone, psuedoephedrine, ranitidine, spironolactone and theophylline.

8. A composition according to any one of the preceding claims, characterized in that it further comprises a flavoring agent.

9. A composition according to claim 8, characterized in that the flavor of the flavoring agent is selected from the group constituted by cherry, strawberry, orange, chocolate, cola, vanilla, grape, mint and lemon.

10. A composition according to any one of the preceding claims, characterized in that it is in the form of a syrup, a suspension, chewing gum, a water-dispersible powder, an effervescent tablet or granules.

11. A composition according to any one of the preceding claims, characterized in that the Albizzia myriophylla bark extract is a dry extract from branch bark.

12. A composition according to any one of the preceding claims, characterized in that the Albizzia myriophylla extract is a protein extract.

13. A composition according to claim 11 or claim 12, characterized in that the Albizzia myriophylla bark extract is obtained by a method comprising the following steps:
a) pulverizing Albizzia myriophylla branch bark;
b) extracting one or more times, the pulverized bark obtained in step a) in a solvent that is miscible with water, advantageously selected from alcohol and water and mixtures thereof in any proportions;
c) filtering the solution obtained in step b) and eliminating the solvent.

14. The use of an Albizzia myriophylla bark extract or a composition according to claims 1 to 13 to suppress or reduce the gustatory function, in particular as regards bitterness and/or a sweet taste and/or an acid taste.

15. The use of an Albizzia myriophylla bark extract or of a composition according to claims 1 to 13, as a hunger suppressant and as a slimming aid or to prevent weight gain.

16. The use of an Albizzia myriophylla bark extract or of a composition according to claims 1 to 13, as an alcohol and tobacco withdrawal aid.

17. A method of reducing or suppressing the taste of a composition, characterized in that it comprises adding an effective quantity of an Albizzia myriophylla bark extract to said composition.

18. A method of reducing or suppressing the gustatory function, in particular as regards a bitter and/or sweet and/or acid taste, characterized in that it comprises oral administration of an effective quantity of an Albizzia myriophylla extract or of a composition according to any one of claims 1 to 13.

19. A method according to claim 18, characterized in that a composition the taste of which is to be reduced or suppressed is administered at the same time or after the Albizzia myriophylla bark extract.

20. An Albizzia myriophylla bark extract and an oral composition according to any one of claims 1 to 13, as a drug.

21. An Albizzia myriophylla bark extract and an oral composition according to any one of claims 1 to 13, as a drug for the treatment of obesity, bulimia, a compulsive eating disorder and alcohol and/or tobacco dependence, and for the prevention of obesity.
